(19) United States
(12) Patent Application Publication
(10) Pub. No.: US 2010/0305435 A1
(43) Pub. Date: Dec. 2, 2010

(54) Bone Marking System and Method

(76) Inventor: John C. Magill, Woburn, MA (US)

Correspondence Address: PROSKAUER ROSE LLP, ONE INTERNATIONAL PLACE, BOSTON, MA, 02110, US

(21) Appl. No.: 12/472,817

(22) Filed: May 27, 2009

(51) Int. Cl. A61B 5/05

(52) U.S. Cl. 600/426

(57) ABSTRACT

An apparatus for marking bone of a patient includes a marker, a tracking system and a display. The marker includes pigment and is configured to impart the pigment on the bone. The tracking system is in communication with the marker. The tracking system is configured to determine a location of the marker relative to the bone and to match at least one anatomical feature of the bone of the patient to an image of the bone. The display is in communication with the tracking system and adapted to show an image of the location of the marker relative to the image of the bone while the marker imparts the pigment on the bone.

### STATEMENT OF GOVERNMENT INTEREST

The subject matter described herein was developed in connection with funding provided by the National Institute of Health, Grant No. 1R43DE019322-01. The Federal government may have rights in the invention.

### FIELD OF THE INVENTION

This invention relates generally to marking bones for surgical procedures, and more particularly to bone surgery wherein a plan for the procedure has been produced on a computer using three-dimensional graphical image display.

### BACKGROUND OF THE INVENTION

Correcting congenital or acquired deformities of the face requires that surgeons consider both functional and aesthetic requirements. Functional requirements include airway, occlusion, expected growth, and others. Aesthetic considerations include bone shape and symmetry, as well as a need to minimize scars from incisions.

Because this type of surgery often involves complex multi-axis manipulations, surgeons have begun to pre-plan procedures using computer-based models generated from computed-tomography scans. It is necessary to transfer these computer-based treatment plans to the patient's anatomy during the operation.

The benefits of guiding instruments, catheters, directed radiation, and other therapeutic tools have already produced several commercial products for surgical navigation. These have been applied to many medical procedures. Prior work and existing products for surgical navigation generally involve one or more of three technologies: real-time imaging, instrument tracking, and merged-image image displays.

Image-guided procedures involve imaging (e.g., fluoroscopy, magnetic resonance imaging, computed-tomography, infrared-light-based techniques and ultrasound) in the operating theater can allow the surgeon to see the location of catheters, biopsy probes, and other instruments. These systems, however, can be expensive, cumbersome, and obstruct the view of a surgeon or his/her assistants.

### SUMMARY OF THE INVENTION

The invention features, in one embodiment, marking bones for surgical procedures (e.g., orthognathic surgery). A bone marking system can enable surgeons to copy computer treatment plans to the bone even through a very small incision, allowing surgeons to perform more sophisticated procedures with less difficulty for the patient and improved outcomes (e.g., faster recovery times, less pain and discomfort, and less scarring than open procedures). A bone marking system can be used in both endoscopic and open procedures to transfer treatment plans from computer-based three dimensional surgical planning tools onto the bone. A system can provide for intraoperative registration of the image to the patient, with no requirement for fiducial markers in the pre-operative images or for a fixation frame attached to the head. The surgeon can mark the bone for ostotomies, screw holes or alignment, and then complete the procedure using instruments without any tracking, using the marks to guide the modification of bone. A bone marking system is less expensive and need not track the motion of multiple surgical instruments as procedures are performed. With operating room costs surpassing $25 per minute, techniques that allow efficient and precise implementation of surgical plans are essential for cost-effective management of surgical cases.

In one embodiment, a bone marking system includes a marking pencil, an electromagnetically-tracked pencil sensor registered to the patient and to the computer model using a registration procedure through a set of anatomical landmarks rigidly fixed to the bone such as points on teeth or features on the bone surface or the surface of a proximate bone that can be seen when the bone is exposed. Once registered, the marking pencil is displayed on the computer image display in a scene containing anatomical structures and indications for bone cuts, screw holes, fixation plates and the like. The marking pencil is an implement for marking said bone cuts, screw holes fixation plates and the like. The image of the marking pencil moves in real time to indicate the position and orientation of the marking pencil relative to the bone of the patient. The surgeon can watch the screen, using the indications on the computer image as a guide for marking the bone. This can be done through a small incision since there is no need physically for line-of-site to see the tip of the pencil. Once the mark(s) are made, the pencil is removed and the procedure is completed using conventional instruments and techniques, either in an open or endoscopic procedure.

The pencil need not be metal, can be tracked electromagnetically rather than optically, removing the line-of-site constraint, and is less intrusive than conventional marking and tracking systems. Because anatomical landmarks rigidly fixed to the bone, such as teeth or features on the bone surface or the surface of a proximate bone, can be used as registration points, no fiducial markers are required when imaging the patient.

The opportunity to minimize incision (and hence scars) is particularly attractive for maxillofacial surgeons, as virtually everyone is sensitive to the appearance of the face. Recovery time and patient discomfort can be reduced when less-invasive techniques are employed. Working through smaller incisions limits the visual field from which surgeons use to manipulate instruments, so new methods of locating instruments with respect to the patient's anatomy are necessary. The bone marking instrument combines minimally invasive therapy with tissue engineering to eliminate donor site morbidity.

The bone marking system can assist in the mapping of a treatment plan from the computer (e.g., a computer-based three-dimensional plan) to the bone structures to be modified. Because orthognathic surgical reconstruction often involves complex multi-axis manipulations, many surgeons find navigation tools helpful even for open procedures. The bone marking system can also be used as a precision measuring device to, for example, establish symmetry in bilateral procedures. Examples of procedures where the bone marking instrument and method might be used include minimally-invasive (endoscopic) reconstruction and distraction osteogenesis.

In one aspect, there is an apparatus for marking bone of a patient. The apparatus includes a marker, a tracking system and a display. The marker includes pigment and is configured to impart the pigment on the bone. The tracking system is in communication with the marker. The tracking system is configured to determine a location of the marker relative to the bone and to match at least one anatomical feature of the bone of the patient to an image of the bone. The display is in communication with the tracking system and adapted to show an image of the location of the marker relative to the image of the bone while the marker imparts the pigment on the bone.

In another aspect, there is a method of marking bone of a patient. The method includes matching at least one anatomical feature of the bone of the patient to an image of the bone, determining a location of a marker comprising pigment relative to the bone, showing an image of the location of the marker relative to the image of the bone on a display, using the display to facilitate positioning the marker relative to a marking locating on the bone, and using the marker to impart the pigment to the marking locating on the bone.

In yet another aspect, there is an apparatus for marking bone of a patient. The apparatus includes means for imparting pigment to a marking location on the bone of the patient, means for matching at least one anatomical feature of the bone of the patient to an image of the bone, means for determining a location of the means for imparting pigment relative to the bone, means for showing an image of the location of the means for imparting pigment relative to the image of the bone and facilitating positioning the means for imparting pigment relative to the marking location on the bone.

In other examples, any of the aspects above, or any apparatus, system or device or any method, process or technique described herein, can include one or more of the following features.

A reference sensor can be in communication with the tracking system and positionable relative to the bone of the patient. The reference sensor can be configured to provide for the tracking system a frame of reference fixed to the bone.

In certain embodiments, the tracking system can include and be in separate communication with a transmitter disposed in a known position relative to the bone of the patient and a receiver attachable to the marker. The receiver can sense a signal from the transmitter to provide location information of the marker so that the tracking system can determine the location of the marker relative to the bone. In certain embodiments, the tracking system can include and be in separate communication with a transmitter attachable to the marker and a receiver disposed in a known position relative to the bone of the patient. The receiver can sense a signal from the transmitter to provide location information of the marker so that the tracking system can determine the location of the marker relative to the bone.

The marker can include a transmitter, disposed in a known position relative to the bone of the patient, providing location information from which a tracking system can determine the location of the marker relative to the bone. The marker can include a receiver, disposed in a known position relative to the bone of the patient, sensing a signal from a transmitter to provide location information of the marker so that a tracking system can determine the location of the marker relative to the bone.

In various embodiments, the at least one anatomical feature can be or can include at least one tooth of the patient, a feature of a surface of the bone, or three anatomical features of the bone. In certain embodiments, the at least one anatomical feature of the bone of the patient can be matched (e.g., by the tracking system) to the image of the bone without exogenous fiducial markers. The location information can include position and orientation of the marker relative to the bone. The receiver and/or the tracking system can sense the position and orientation of the marker relative to the bone. The marker can be a pencil.

In certain embodiments, the method includes obtaining the image of the bone prior to matching the at least one anatomical feature of the bone of the patient to the image of the bone, developing a planned surgical procedure based on the image obtained, and performing the surgical procedure to the bone after imparting color to the marking locating on the bone.

Other aspects and advantages of the invention will become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a depiction of a bone marking instrument.

FIG. 2 is another depiction of a bone marking instrument.

FIG. 3 is another depiction of a bone marking instrument.

FIGS. 4A-4C shows various markers.

FIG. 5 is a depiction of a registration process.

FIG. 6 is a series of geometric transformations that are computed to relate the patient reference frame to the computer model.

FIG. 7 is a flow diagram showing the process for registering an image and marking bone.

FIG. 8 is a depiction of bone marking instrument used in knee replacement surgery.

### DESCRIPTION OF THE INVENTION

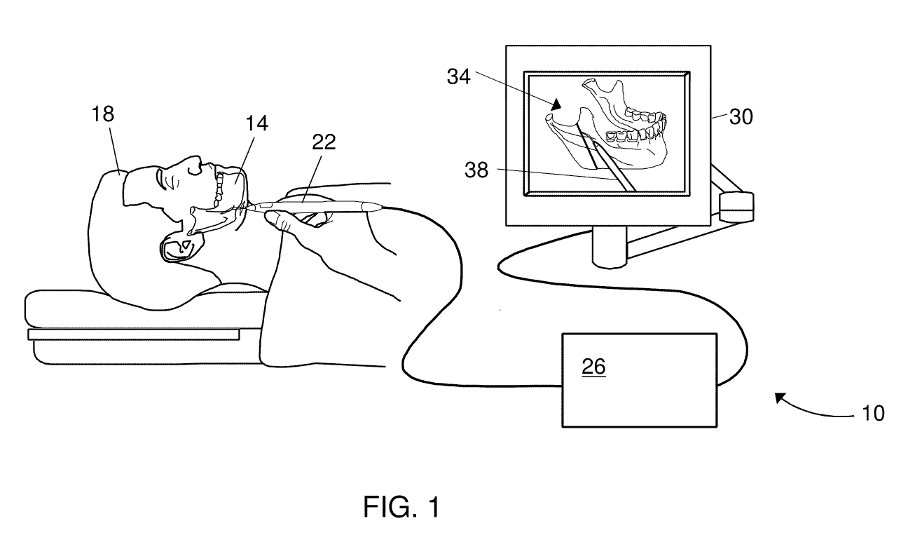

FIG. 1 shows an apparatus 10 for marking a bone 14 of a patient 18. The bone marking apparatus 10 includes a marker 22, a tracking system 26, and a display 30. The marker 22 includes pigment and can be configured to impart the pigment on the bone. The tracking system 26 is in communication with the marker 22. The tracking system 26 can be configured to determine a location of the marker 22 relative to the bone 14 and configured to match at least one anatomical feature of the bone 14 to an image 34 of the bone. The display 30 is in communication with the tracking system 26 and is configured to show an image 38 of the location of the marker 22 relative to the image 34 of the bone while the marker 22 imparts the pigment on the bone 14. The bone marking apparatus 10 can include a reference sensor to provide for the tracking system a frame of reference fixed to the bone. The marker 22 can be an implement for marking bone cuts, screw holes, fixation plates and the like.

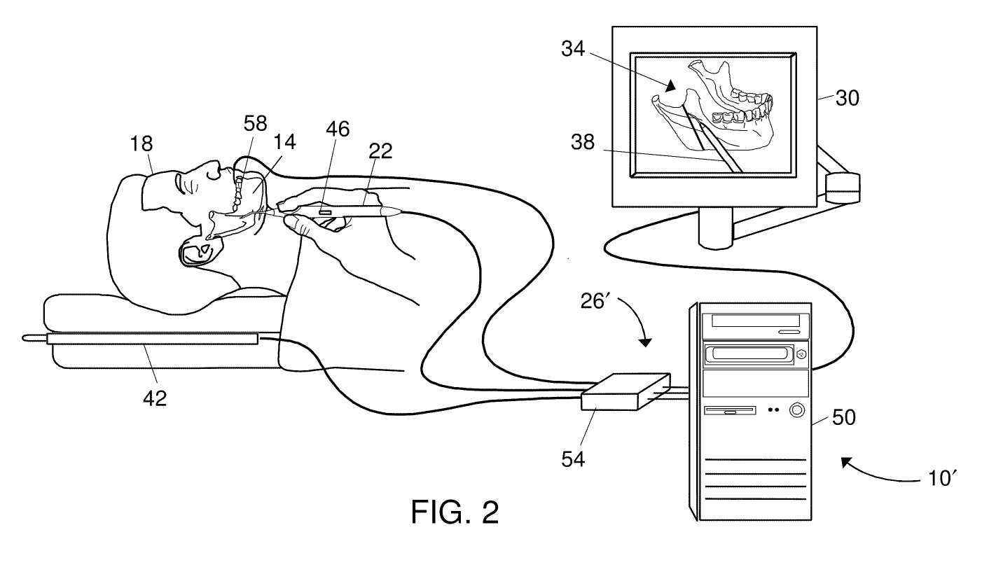

FIG. 2 shows another embodiment of a bone marking instrument 10′. The tracking system 26′ includes and is in communication with a transmitter 42 disposed relative to the bone 14 of the patient 18 and a receiver 46 attachable to the marker 22. The transmitter 42 can be disposed in a known position relative to the bone 14 of the patient. The receiver 46 senses a signal from the transmitter 42 to provide location information of the marker 22 so that the tracking system 26′ can determine the location of the marker 22 relative to the bone 14. The tracking system 26′ can include a computer controller 50 and a tracking interface 54, which can be in communication with the transmitter 42, the receiver 46 and a reference sensor 58. Display 26 can show an image 38 of the location of the marker 22 relative to the image 34 of the bone. The reference sensor 58 can provide for the tracking system 26′ a frame of reference fixed to the bone 14.

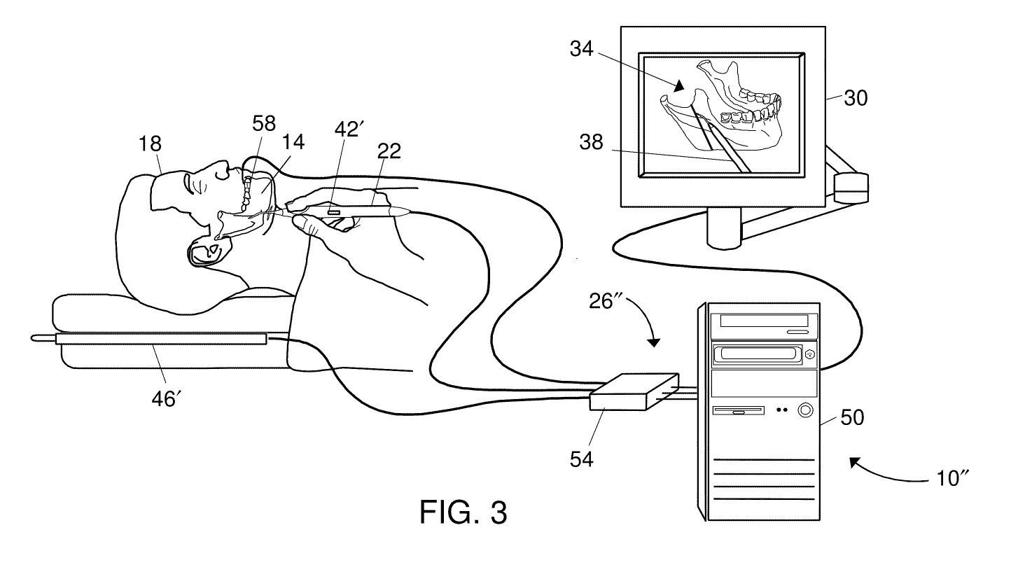

FIG. 3 shows another embodiment of a bone marking instrument 10″. The tracking system 26″ includes and is in communication with a transmitter 42′ attachable to the marker 22 and a receiver 46′ disposed relative to the bone 14 of the patient 18. The receiver 46′ can be disposed in a known position relative to the bone 14 of the patient. The receiver 46′ senses a signal from the transmitter 42′ to provide location information of the marker 22 so that the tracking system 26″ can determine the location of the marker 22 relative to the bone 14. The tracking system 26″ can include a computer controller 50 and a tracking interface 54, which can be in communication with the transmitter 42′, the receiver 46′ and a reference sensor 58. Display 26 can show an image 38 of the location of the marker 22 relative to the image 34 of the bone. The reference sensor 58 can provide for the tracking system 26″ a frame of reference fixed to the bone 14.

The marker 22 can include or contain a pigment, and can be configured to impart the pigment on the bone. The marker can be a pencil, a pen or other marking instrument. The pigment can be a pencil lead, graphite, ink, dye, or other suitable markant. The pigment can be a solid, or a fluid or gel contained within a vessel. In certain embodiments, the marker is a #2 pencil that has been sterilized prior to being used in a bone marking procedure.

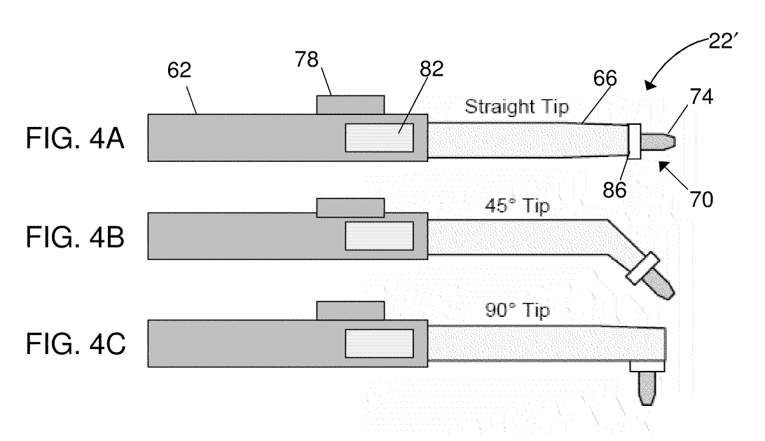

FIGS. 4A-4C shows various markers 22′. As illustrated, the markers 22′ include a body 62, an extension member 66, and a tip 70, which can include a pigment 74. The body 62 can include a button 78 and a transmitter/receiver 82, although these features can be included on the extension member 66. The body 62 can serve as a holder for the markers 22′, and can be manipulated by a surgeon. The body 62 can define a bore, into which the extension member 66 is insertable. For example, the body 62 can have a threaded bore into which the extension member 66, having a threaded end, engages to form a thread lock.

The extension member 66 can define a bore, into which the tip 70 is insertable. For example, the extension member 66 can have a threaded bore into which the tip 70, having a threaded end, engages to form a thread lock. The tip 70 can be entirely composed of the pigment 74, or can include a holder portion 86 for the pigment 74. In certain embodiments, tip 70 can be a disposable so that it can be replaced when the pigment is consumed, or between surgical procedures on different patients. In certain embodiments, the extension member 66 and the tip 70 can be one contiguous body, which is insertable in the bore of the body 62. For example, the extension member 66 and the tip 70 can be a pencil that is insertable into the body 62.

The body 62 and/or the extension member 66 can be formed from a glass-reinforced material or a plastic (e.g., poly-ether-ether-ketone (PEEK)). The holder portion 86 of the tip 70 can be nylon and/or threaded. For example, the pigment can thread into a nylon holder. The body 62 and/or the extension member 66 can be straight or formed at an angle with respect to its longitudinal axis. FIG. 4A shows an embodiment where the extension member 66 is straight or substantially straight. FIG. 4B and 4C show embodiments where the extension member 66 is bent (e.g., at a 45° or 90° angle, although any angle can be achieved).

The tip 70 can be configured to make indentations or pilot holes in the bone. For example, the tip 70 can be like the point of an awl. The button 78 or switch can be used to indicate when the marker 22′ is in position for a registration point, and/or can be used when marking to indicate to the graphics program to draw on the display screen.

The marker 22 or 22′ can be sufficiently rigid so that placement of the tip can be repeatable. For example, the marker can provide less than 1 millimeter of error under modest pressure when flexed. The software can be programmed to assume a tip location with respect to the transmitter/receiver 82 so that deflection to another point is undesirable. In certain embodiments, the sensor can be in the tip.

The transmitter/receiver 82 can be one of transmitter 42, transmitter 42′, receiver 46, or receiver 46′. In certain embodiments, the transmitter/receiver 82 can include more than one transmitter or receiver. For example, a second receiver can be included in the marker 22. The tracking system 26 can make orientation measurements at two different axial locations of the marker 22. This can improve accuracy, for example, by accounting for flexing of the marker 22 when downward pressure is applied by a surgeon. The two axial locations can account for the degree of bending of the marker 22, which can be used to more accurately determine the tip location. Furthermore, averaging signals from the two sensors can reduce random noise sources.

The marker 22 can have an ergonomic design for comfort and ease of use by a surgeon. Additional buttons can be on the body of the marker for added functionality.

The tracking system 26 can be a 3D Guidance medSAFE electromagnetic tracking system (available from Ascension Technology, Burlington, Vt.) including a flat antenna and receiver sensors. The flat antenna can be placed under a patient's head and transmit gradients in generated electric and magnetic fields to determine the location and orientation of sensors (e.g., a receiver in the marker and a reference sensor) within the fields.

The tracking interface 54 can be a control module of the 3D Guidance medSAFE electromagnetic tracking system. The tracking interface 54 can be coupled to the antenna, receiver sensors, and the reference sensor via cables, and can be coupled to the computer controller 50 via a USB port. In certain embodiments, the tracking interface 54 can communicate with a transmitter, receiver, sensor, or computer processor wirelessly. In certain embodiments, the tracking interface 54 is a component of the computer controller 50.

The tracking interface 54 can be configured to determine a location of the marker 22 relative to the bone 14. For example, the tracking interface 54 can generate a signal to initiate the electric and magnetic fields generated by the antenna/transmitter, and the tracking interface can receive a signal from the receiver/sensor. The tracking interface 54 can calculate location information (e.g., position and orientation) of the marker based on the signal(s) received. The tracking interface 54 can communicate the location information to the computer controller 50. A controller or computer processor running software can perform these functions.

The display 30 can be, for example, a cathode ray tube (CRT), a liquid crystal display (LCD) monitor or a projection device. The display 30 can show, depict, project and/or superimpose an image of the marker 22 on or relative to an image of the bone on the display. A surgeon can view the display 30 while marking the bone according to a pre-operative, computer-based marking plan.

The reference sensor 58 can be positionable on a tooth or on bone surface. The reference sensor 58 can be placed on, affixed to, clamped on, glued on, and/or bonded to the tooth or bone surface. For example, a holder can be bonded to a tooth with a UV-curing dental adhesive. The holder can be disposable. The reference sensor can be bonded into a carrier that snaps into the holder. The adhesive can be removed without damaging the tooth at the conclusion of the procedure. In certain embodiments, the reference sensor can be a dental splint or bite held in place by the patient.

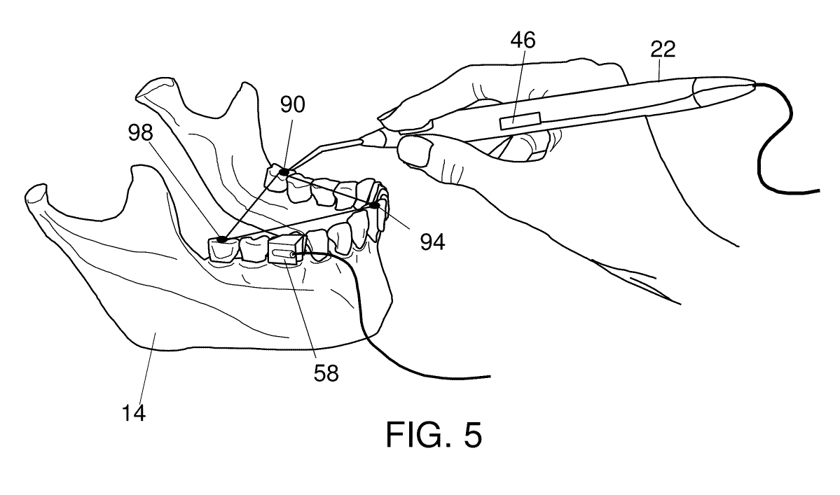

FIG. 5 shows an exemplary process of registration for oral surgery. Registration can match at least one anatomical feature of the bone of the patient to an image of the bone. The anatomical feature(s) can be a tooth/teeth, a feature of a surface of the bone, and/or three anatomical features of the bone (e.g., three teeth). FIG. 5 shows three teeth as registration points 90, 94 and 98. The tracking system can be adapted to match the anatomical feature(s) of the bone of the patient to the image of the bone without exogenous fiducial markers. The image registration process can include an initial registration process using at least three registration points followed by a second registration process (e.g., using three additional registration points).

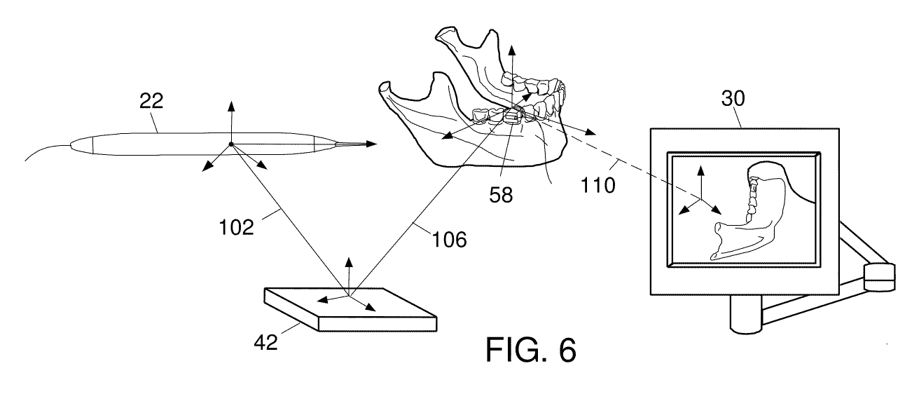

The registration algorithm can relate the patient-fixed reference frame to the coordinates of the computer model derived from the patient's 3D image scan, as illustrated in FIG. 6. The relationship is represented by a rotation and an offset. Once this relationship has been determined, the bone marker is located with respect to the patient reference frame, and this relationship is image displayed visually on the image display. To locate the marker with respect to the patient, both are tracked in the reference frame of the receiver.

The general form of the coordinate transformation of a point V from frame A to frame B is:

V^{B}=R_{A}^{B}V^{A}+X_{A}^{B}

where the matrix R is the rotation matrix and X is the offset. To image display the marker in the ‘M’ reference frame, a sequence of known and measured offsets and rotations is applied. As shown in FIG. 6, with tip ‘t’ of marker within the frame ‘P’ of the transmitter, a first geometric transformation 102 of transmitter ‘T’ frame, a second geometric transformation 106 of reference sensor ‘R’ frame, and a third geometric transformation 110 to the image frame ‘M’ are stepped through. The equation is:

Xₜ^{M}=R_{R}^{M}(R_{T}^{R}((R_{P}^{T}Xₜ^{P}+Xₚ^{T})+Xᵣ^{T})Xₘ^{R})

There are seven terms required to compute Xₜ^{M}. Xₜ^{P} is fixed and represents the location of the marker tip with respect to the marker. R_{P}^{T} and R_{R}^{T} and the companion origin offset vectors Xₚ^{T} and Xᵣ^{T} are supplied by the tracker at each update. The rotation R_{R}^{M} and offset Xᵣ^{M} are computed in the image registration process of the registration algorithm.

To determine R_{R}^{M} and offset Xᵣ^{M}, three points can be located, e.g., first registration point 90, second registration point 94 and third registration point 98, as shown in FIG. 5, in both the computer image and the patient. Four points can be used to determine a coordinate transformation in 3D. Since a mirror-image transformation between the computer image and the patient is not being used, three points can be used.

Generally, the greater the number of registration points, the more accurate the image registration process is. The N points used for registration are designated P_{I} through P_{N}. The measurements can be acquired in transmitter coordinates and converted to reference-sensor coordinates since the necessary offsets and rotations can be continually measured. Note that the patient's head can be moved between points to access them, so a different set of rotations and offsets can be measured for each. Once the tip of the marker is in contact with a registration point, a key or button is pressed to prompt the software to make a measurement.

For the i^{th} point, R_{P}^{T}, R_{R}^{T}, Xₚ^{T} and Xᵣ^{T} are measured. Then, the following is computed:

Xₜ^{R}=R_{T}^{R}(R_{P}^{T}Xₜ^{P}+X_{P}^{T})+Xᵣ^{T}

which represents (Pᵢ)^{R}, the location of the i^{th} registration point in the frame of the reference sensor. These points are also known in the model (M) reference frame.

Using the more compact form:

$${P}^{M} = [ \begin{matrix}{R}_{R}^{M} & {X}_{R}^{M}\end{matrix} ] [ \begin{matrix}{P}^{R} \\ 1\end{matrix} ]$$

R_{R}^{M} and Xᵣ^{M} are computed. The matrices are constructed:

$$G = {[ \begin{matrix}{P}_{1}^{R} & & {P}_{N}^{R} \\ & … & \\ 1 & & 1\end{matrix} ]}^{T}$$

### and H=[P₁^{M} . . . P_{N}^{M}]^{T}.

and solved for W=[R_{R}^{M} Xᵣ^{M}] from H=WG using least-squares:

W^{T}=(G^{T}G)⁻¹G^{T}H.

Now, from W, R_{R}^{M} (the first three columns) is extracted, and X_{R}^{M} (the last column). As shown in FIG. 5, registration can be performed using an initial registration with three points (e.g., first registration point 90, second registration point 94 and third registration point 98). At this point, the computer can image display the image of the moving marker on the image display. The user can touch the marker to several points on the bone, identifying places where there is an error between the physical position and that shown on the image display. These are then selected as additional registration points (typically, 1-3 additional registration points. The first registration point 90, the second registration point 94 and the third registration point 98 are generally not re-registered because the driver stores these registration points, and re-computes the rotation and offset each time a new point is added.

After the bone of interest has been exposed, the surgeon can mark (precisely or substantially precisely) locations for screw pilot holes, osteotomies, plates, distractors, and the like with the aid of image display of the marker and image of the bone in the operating theater. The surgeon can complete the surgical procedure with conventional instruments, using the bone marks as guides.

The bone marking system can be used to transfer treatment plans from computer-based 3D surgical planning tools onto the bone. The bone marking system includes a marker that is tracked electronically and displayed with respect to a 3D image of patient anatomy. Prior to a surgical procedure, a surgeon can create the image from CT scans (or other similar measurements), design the reconstruction of the bone, and indicate on the computer model locations for plates, screws, osteotomies and other entities. During surgery, watching the marker on the display, the surgeon can mark the bone to indicate the desired locations of the key entities, and then complete the procedure with conventional instruments.

The surgical procedure can be a planned osteotomy, e.g., a minimally invasive endoscopic reconstruction surgery, an orthognathic reconstruction surgery, a knee replacement surgery, for marking the location of screw pilot holes, for marking the location of plates, or for marking the location of distractors.

Surgical procedures can include, but are not limited to, orthognathic surgery including endoscopic procedures, correcting improper alignment and position of the jaw bone, LeFort I osteotomy, bilateral saggital split osteotomy, vertical ramus osteotomy, and distraction osteogenesis (e.g., identifying device placement, vector of distraction, osteotomy, and screw holes); surgery for trauma and reconstruction, including fragment and fixation hardware placement for orbital floor, zygomatic bone, NOE, mandible, and the like; pathology including tumor resection (identifying extent in bone), reconstruction, and bioposy; temporo-mandibular joint procedures including release of ankylosis, condylectomy and prosthesis placement; dental implants including bone graft, sinus augmentation and implant placement; pre-prosthetic surgery including bone grafts; and cosmetic surgery including implant placement and genioplasty (chin).

A bone marking system can also be used to measure distances for pre-planning and for intra-operative planning and decision-making. Even without pre-planning, symmetry and accuracy of measurements can be assured.

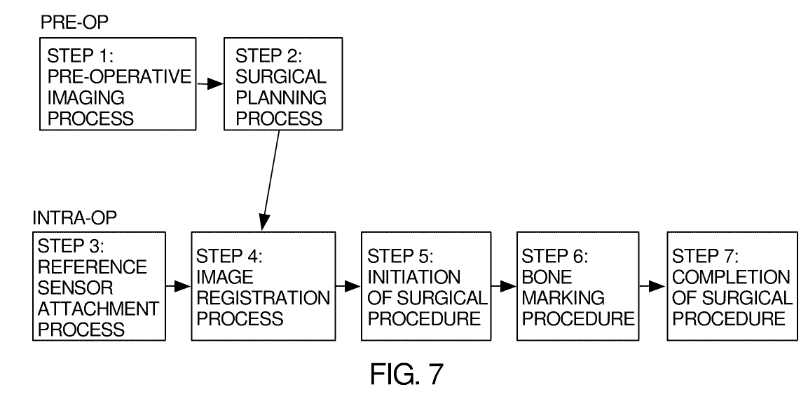

FIG. 7 shows process flow for marking a bone and performing a surgical procedure, including:

Step 1. Pre-operative Imaging Process—the surgeon obtains a pre-operative image using 3D computer images of the operative area of the patient taken using CT or other three dimensional imaging technology.

Step 2. Surgical Planning Process—the surgeon develops a planned osteotomy based on the pre-operative imaging for the surgical corrections to bone structures.

Step 3. Reference Sensor Attachment Process—the surgeon rigidly affixes a reference sensor to an anatomical landmark, such as teeth or features on the bone surface or the surface of a proximate bone.

Step 4. Image Registration Process—the surgeon registers the patient to the pre-operative images using a registration algorithm and the marker.

Step 5. Initiation of Surgical Procedure—the surgeon initiates the planned osteotomy by making an incision sufficient for the marker to penetrate to the bone.

Step 6. Bone Marking Procedure—the surgeon uses the marker to mark the bone to indicate the planned osteotomy.

Step 7. Completion of Surgical Procedure—the surgeon completes the surgical procedure using conventional, surgical tools.

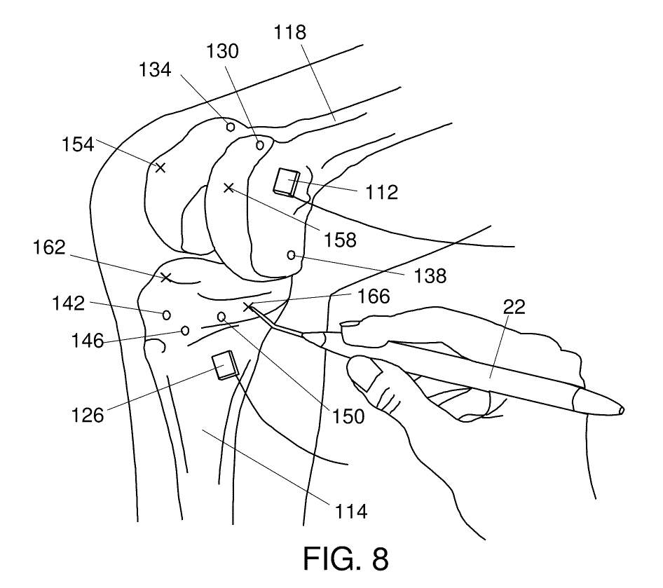

FIG. 8 shows another embodiment of the bone marking instrument for knee replacement surgery or knee arthroplasty. Alignment of implant hardware for a tibia 114 and femur 118 is critical to implant success and longevity. A template is attached to the bone to guide cutting and drilling of the bone to accept the implant hardware. This alignment is usually done visually by the surgeon. Using a bone marking system, a femur reference sensor 122 is attached to the femur 118 and a tibia reference sensor 126 is attached to the tibia 114. A first femur registration point 130 and second femur registration point 134 and third femur registration point 138 is associated with the femur, and a first tibia registration point 142 and second registration point 146 and third registration point 150 is associated with the tibia. Using the process described above, the tibia 114 and the femur 118 are registered to their respective images. The marker 22 is then used to precisely place a first femur reference mark 154 and a second femur reference mark 158 on the femur 118, and similarly, the marker 22 is then used to precisely place a first tibia reference mark 162 and a second tibia reference mark 166 on the tibia 114 to indicate proper template position.

The above-described systems and methods can be implemented in digital electronic circuitry, in computer hardware, firmware, and/or software. The implementation can be as a computer program product (i.e., a computer program tangibly embodied in a computer-readable storage device). The implementation can, for example, be in a machine-readable storage device for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose circuitry. The circuitry can, for example, be a FPGA (field programmable gate array), an ASIC (application-specific integrated circuit), or the like. Modules refer to portions of the hardware (e.g., the processor, processor and memory, the special circuitry and the like), including the software elements (e.g., computer program), that implements that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more computer readable storage devices for storing data (e.g., magnetic, magneto-optical disks, or optical disks).

Data transmission and instructions can also occur over a communications network. Information carriers suitable for embodying computer program instructions and data include computer readable storage mediums, for example all forms of non-volatile memory, including by way of example semiconductor memory devices. The computer readable storage mediums can, for example, be EPROM, EEPROM, flash memory devices, magnetic disks, internal hard disks, removable disks, magneto-optical disks, CD-ROM, and/or DVD-ROM disks. The processor and the memory can be supplemented by and/or incorporated in special purpose logic circuitry.

To provide for interaction with a user, the above described techniques can be implemented on a computer having a display device or a transmitting device. The display device can be, for example, a cathode ray tube (CRT), a liquid crystal display (LCD) monitor and/or a projection device. The interaction with a user can be, for example, a display of information to the user and a keyboard and a pointing device (e.g., a mouse or a trackball) by which the user can provide input to the computer (e.g., interact with a user interface element). Other kinds of devices can be used to provide for interaction with a user. Other devices can be, for example, feedback provided to the user in any form of sensory feedback (e.g., visual feedback, auditory feedback, or tactile feedback). Input from the user can be, for example, received in any form, including acoustic, speech, and/or tactile input.

The computing device can include, for example, a computer, a computer with a browser device, a telephone, an IP phone, a mobile device (e.g., cellular phone, personal digital assistant (PDA) device, laptop computer, electronic mail device), a server, a rack with one or more processing cards, special purpose circuitry, and/or other communication devices. The browser device includes, for example, a computer (e.g., desktop computer, laptop computer) with a World Wide Web browser (e.g., Microsoft® Internet Explorer® available from Microsoft® Corporation, Mozilla® Firefox available from Mozilla® Corporation). The mobile computing device includes, for example, a Blackberry® or iPHONE®.

The web servers can be, for example, a computer with a server module (e.g., Microsoft® Internet Information Services available from Microsoft® Corporation, Apache Web Server available from Apache Software Foundation, Apache Tomcat Web Server available from Apache Software Foundation).

The databases can be, for example, a computer with a server module (e.g., Microsoft® SQL Server 2008 available from Microsoft® Corporation and/or Oracle® Database 11g available from Oracle® Corporation).

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network).

The system can include clients and servers. A client and a server are generally remote from each other and typically interact through a communication network. The relationship of client and server arises by virtue of computer programs running on the respective computers and having a client-server relationship to each other.

The above described communications networks can be implemented in a packet-based network, a circuit-based network, and/or a combination of a packet-based network and a circuit-based network. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, Bluetooth, code-division multiple access (CDMA) network, time division multiple access (TDMA) network, global system for mobile communications (GSM) network), and/or other circuit-based networks.

Comprise, include, and/or plural forms of each are open ended and include the listed parts and can include additional parts that are not listed. And/or is open ended and includes one or more of the listed parts and combinations of the listed parts.

Although various aspects of the disclosed instrument and method have been shown and described, modifications may occur to those skilled in the art upon reading the specification. The present application includes such modifications and is limited only by the scope of the claims, and all changes that come within the meaning and range of equivalency of the claims are therefore intended to be embraced therein.

What is claimed is:

1. An apparatus for marking bone of a patient, comprising:
a marker comprising pigment and being configured to impart the pigment on the bone;
a tracking system in communication with the marker, configured to determine a location of the marker relative to the bone, and configured to match at least one anatomical feature of the bone of the patient to an image of the bone; and
a display in communication with the tracking system and adapted to show an image of the location of the marker relative to the image of the bone while the marker imparts the pigment on the bone.

2. The apparatus of claim 1 further comprising a reference sensor in communication with the tracking system and positionable relative to the bone of the patient, the reference sensor configured to provide for the tracking system a frame of reference fixed to the bone.

3. The apparatus of claim 1 wherein the tracking system comprises and is in separate communication with:
a transmitter disposed in a known position relative to the bone of the patient; and
a receiver attachable to the marker, the receiver sensing a signal from the transmitter to provide location information of the marker so that the tracking system can determine the location of the marker relative to the bone.

4. The apparatus of claim 1 wherein the tracking system comprises and is in separate communication with:
a transmitter attachable to the marker; and
a receiver disposed in a known position relative to the bone of the patient, the receiver sensing a signal from the transmitter to provide location information of the marker so that the tracking system can determine the location of the marker relative to the bone.

5. The apparatus of claim 1 wherein the at least one anatomical feature includes at least one tooth of the patient.

6. The apparatus of claim 1 wherein the at least one anatomical feature includes a feature of a surface of the bone.

7. The apparatus of claim 1 wherein the at least one anatomical feature includes three anatomical features of the bone.

8. The apparatus of claim 1 wherein the tracking system is adapted to match the at least one anatomical feature of the bone of the patient to the image of the bone without exogenous fiducial markers.

9. The apparatus of claim 1 wherein the tracking system determines location from location information including position and orientation of the marker relative to the bone.

10. The apparatus of claim 1 wherein the marker is a pencil.

11. A method of marking a bone of a patient, comprising:
matching at least one anatomical feature of the bone of the patient to an image of the bone;
determining a location of a marker comprising pigment relative to the bone;
showing an image of the location of the marker relative to the image of the bone on a display;
using the display to facilitate positioning the marker relative to a marking locating on the bone; and
using the marker to impart the pigment to the marking locating on the bone.

12. The method of claim 11 further comprising using a reference sensor to provide for a tracking system a frame of reference fixed to the bone for matching the at least one anatomical feature of the bone of the patient to the image of the bone.

13. The method of claim 11 wherein the at least one anatomical feature includes at least one tooth of the patient.

14. The method of claim 11 wherein the at least one anatomical feature includes a feature of a surface of the bone.

15. The method of claim 11 wherein the at least one anatomical feature includes three anatomical features of the bone.

16. The method of claim 11 further comprising matching the at least one anatomical feature of the bone of the patient to the image of the bone without exogenous fiducial markers.

17. The method of claim 11 wherein the marker comprises a transmitter, disposed in a known position relative to the bone of the patient, providing location information from which a tracking system can determine the location of the marker relative to the bone.

18. The method of claim 11 wherein the marker comprises a receiver, disposed in a known position relative to the bone of the patient, sensing a signal from a transmitter to provide location information of the marker so that a tracking system can determine the location of the marker relative to the bone.

19. The method of claim 11 further comprising sensing position and orientation of the marker relative to the bone.

20. The method of claim 10 further comprising:
obtaining the image of the bone prior to matching the at least one anatomical feature of the bone of the patient to the image of the bone;
developing a planned surgical procedure based on the image obtained; and
performing the surgical procedure to the bone after imparting color to the marking locating on the bone.

21. An apparatus for marking bone of a patient, comprising:
means for imparting pigment to a marking location on the bone of the patient;
means for matching at least one anatomical feature of the bone of the patient to an image of the bone;
means for determining a location of the means for imparting pigment relative to the bone; and
means for showing an image of the location of the means for imparting pigment relative to the image of the bone and facilitating positioning the means for imparting pigment relative to the marking location on the bone.
